(19) United States
(12) Patent Application Publication
(10) Pub. No.: US 2003/0128848 A1
(43) Pub. Date: Jul. 10, 2003

(54) Method and apparatus for removing noise from electronic signals

(76) Inventor: Gregory C. Burnett, Livermore, CA (US)

Correspondence Address: Shemwell Gregory & Courtney LLP, Suite 201, 4880 Stevens Creek Boulevard, San Jose, CA, 95129, US

(21) Appl. No.: 10/301,237

(22) Filed: Nov. 21, 2002

Related U.S. Application Data
(63) Continuation-in-part of application No. 09/905,361, filed on Jul. 12, 2001.

(51) Int. Cl. A61F 11/06; G10K 11/16; H03B 29/00

(52) U.S. Cl. 381/71.8; 381/71.14

(57) ABSTRACT

A method and system for removing acoustic noise removal from human speech is described. Acoustic noise is removed regardless of noise type, amplitude, or orientation. The system includes a processor coupled among microphones and a voice activation detection (“VAD”) element. The processor executes denoising algorithms that generate transfer functions. The processor receives acoustic data from the microphones and data from the VAD. The processor generates various transfer functions when the VAD indicates voicing activity and when the VAD indicates no voicing activity. The transfer functions are used to generate a denoised data stream.

### RELATED APPLICATIONS

This patent application is a continuation in part of U.S. patent application Ser. No. 09/905,361, filed Jul. 12, 2001, which is hereby incorporated by reference. This patent application also claims priority from U.S. Provisional Patent Application Serial No. 60/332,202, filed Nov. 21, 2001.

### FIELD OF THE INVENTION

The invention is in the field of mathematical methods and electronic systems for removing or suppressing undesired acoustical noise from acoustic transmissions or recordings.

### BACKGROUND

In a typical acoustic application, speech from a human user is recorded or stored and transmitted to a receiver in a different location. In the environment of the user, there may exist one or more noise sources that pollute the signal of interest (the user's speech) with unwanted acoustic noise. This makes it difficult or impossible for the receiver, whether human or machine, to understand the user's speech. This is especially problematic now with the proliferation of portable communication devices like cellular telephones and personal digital assistants. There are existing methods for suppressing these noise additions, but they have significant disadvantages. For example, existing methods are slow because of the computing time required. Existing methods may also require cumbersome hardware, unacceptably distort the signal of interest, or have such poor performance that they are not useful. Many of these existing methods are described in textbooks such as “Advanced Digital Signal Processing and Noise Reduction” by Vaseghi, ISBN 0-471-62692-9.

### BRIEF DESCRIPTION OF THE FIGURES

FIG. 1 is a block diagram of a denoising system, under an embodiment.

FIG. 2 is a block diagram illustrating a noise removal algorithm, under an embodiment assuming a single noise source and a direct path to the microphones.

FIG. 3 is a block diagram illustrating a front end of a noise removal algorithm of an embodiment generalized to n distinct noise sources (these noise sources may be reflections or echoes of one another).

FIG. 4 is a block diagram illustrating a front end of a noise removal algorithm of an embodiment in a general case where there are n distinct noise sources and signal reflections.

FIG. 5 is a flow diagram of a denoising method, under an embodiment.

FIG. 6 shows results of a noise suppression algorithm of an embodiment for an American English female speaker in the presence of airport terminal noise that includes many other human speakers and public announcements.

FIG. 7 is a block diagram of a physical configuration for denoising using unidirectional and omnidirectional microphones, under the embodiments of FIGS. 2, 3, and 4.

FIG. 8 is a denoising microphone configuration including two omnidirectional microphones, under an embodiment.

FIG. 9 is a plot of the C required versus distance, under the embodiment of FIG. 8.

FIG. 10 is a block diagram of a front end of a noise removal algorithm under an embodiment in which the two microphones have different response characteristics.

FIG. 11A is a plot of the difference in frequency response (percent) between the microphones (at a distance of 4 centimeters) before compensation.

FIG. 11B is a plot of the difference in frequency response (percent) between the microphones (at a distance of 4 centimeters) after DFT compensation, under an embodiment.

FIG. 11C is a plot of the difference in frequency response (percent) between the microphones (at a distance of 4 centimeters) after time-domain filter compensation, under an alternate embodiment.

### DETAILED DESCRIPTION

The following description provides specific details for a thorough understanding of, and enabling description for, embodiments of the invention. However, one skilled in the art will understand that the invention may be practiced without these details. In other instances, well-known structures and functions have not been shown or described in detail to avoid unnecessarily obscuring the description of the embodiments of the invention.

Unless described otherwise below, the construction and operation of the various blocks shown in the figures are of conventional design. As a result, such blocks need not be described in further detail herein, because they will be understood by those skilled in the relevant art. Such further detail is omitted for brevity and so as not to obscure the detailed description of the invention. Any modifications necessary to the blocks in the Figures (or other embodiments) can be readily made by one skilled in the relevant art based on the detailed description provided herein.

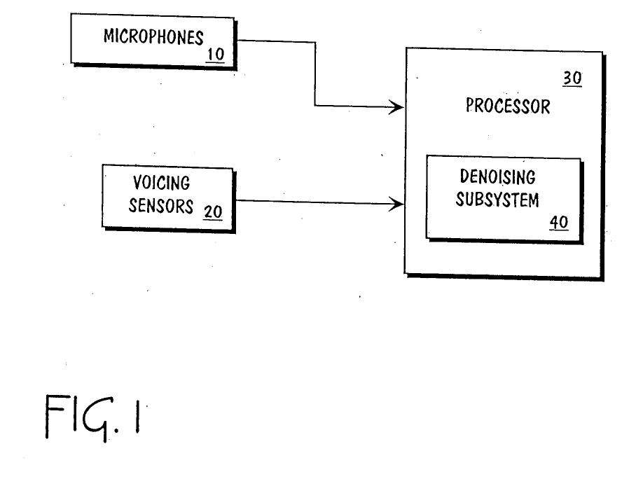

FIG. 1 is a block diagram of a denoising system of an embodiment that uses knowledge of when speech is occurring derived from physiological information on voicing activity. The system includes microphones 10 and sensors 20 that provide signals to at least one processor 30. The processor includes a denoising subsystem or algorithm 40.

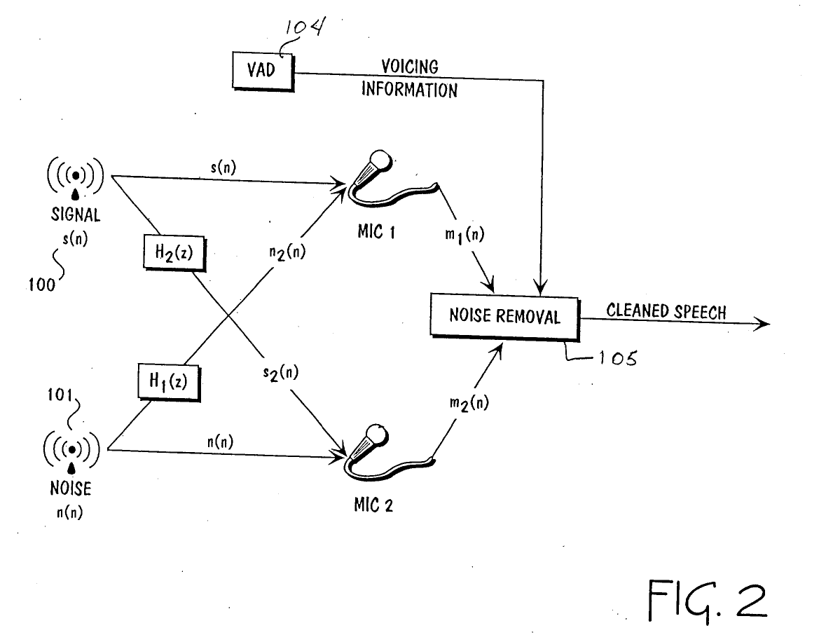

FIG. 2 is a block diagram illustrating a noise removal algorithm of an embodiment, showing system components used. A single noise source and a direct path to the microphones are assumed. FIG. 2 includes a graphic description of the process of an embodiment, with a single signal source 100 and a single noise source 101. This algorithm uses two microphones: a “signal” microphone 1 (“MIC1”) and a “noise” microphone 2 (“MIC 2”), but is not so limited. MIC 1 is assumed to capture mostly signal with some noise, while MIC 2 captures mostly noise with some signal. The data from the signal source 100 to MIC 1 is denoted by s(n), where s(n) is a discrete sample of the analog signal from the source 100. The data from the signal source 100 to MIC 2 is denoted by s₂(n). The data from the noise source 101 to MIC 2 is denoted by n(n). The data from the noise source 101 to MIC 1 is denoted by n₂(n). Similarly, the data from MIC 1 to noise removal element 105 is denoted by m₁(n), and the data from MIC 2 to noise removal element 105 is denoted by m₂(n).

The noise removal element also receives a signal from a voice activity detection (“VAD”) element 104. The VAD 104 detects uses physiological information to determine when a speaker is speaking. In various embodiments, the VAD includes a radio frequency device, an electroglottograph, an ultrasound device, an acoustic throat microphone, and/or an airflow detector.

The transfer functions from the signal source 100 to MIC 1 and from the noise source 101 to MIC 2 are assumed to be unity. The transfer function from the signal source 100 to MIC 2 is denoted by H₂(z), and the transfer function from the noise source 101 to MIC 1 is denoted by H₁(z). The assumption of unity transfer functions does not inhibit the generality of this algorithm, as the actual relations between the signal, noise, and microphones are simply ratios and the ratios are redefined in this manner for simplicity.

In conventional noise removal systems, the information from MIC 2 is used to attempt to remove noise from MIC 1. However, an unspoken assumption is that the VAD element 104 is never perfect, and thus the denoising must be performed cautiously, so as not to remove too much of the signal along with the noise. However, if the VAD 104 is assumed to be perfect such that it is equal to zero when there is no speech being produced by the user, and equal to one when speech is produced, a substantial improvement in the noise removal can be made.

In analyzing the single noise source 101 and the direct path to the microphones, with reference to FIG. 2, the total acoustic information coming into MIC 1 is denoted by m₁(n). The total acoustic information coming into MIC 2 is similarly labeled m₂(n). In the z (digital frequency) domain, these are represented as M₁(z) and M₂(z). Then

M₁(z)=S(z)+N₂(z)

M₂(z)=N(z)+S₂(z)

with

N₂(z)=N(z)H₁(z)

S₂(z)=S(z)H₂(z)

so that

M₁(z)=S(z)+N(z)H₁(z)

M₂(z)=N(z)+S(z)H₂(z) Eq. 1

This is the general case for all two microphone systems. In a practical system there is always going to be some leakage of noise into MIC 1, and some leakage of signal into MIC 2. Equation 1 has four unknowns and only two known relationships and therefore cannot be solved explicitly.

However, there is another way to solve for some of the unknowns in Equation 1. The analysis starts with an examination of the case where the signal is not being generated, that is, where a signal from the VAD element 104 equals zero and speech is not being produced. In this case, s(n)=S(z)=0, and Equation 1 reduces to

M₁ₙ(z)=N(z)H₁(z)

M₂ₙ(z)=N(z)

where the n subscript on the M variables indicate that only noise is being received. This leads to

$${M}_{1 n} ( z ) = {M}_{2 n} ( z ) {H}_{1} ( z ) \\ {H}_{1} ( z ) = \frac{{M}_{1 n} ( z )}{{M}_{2 n} ( z )} \qquad Eq . 2$$

H₁(z) can be calculated using any of the available system identification algorithms and the microphone outputs when the system is certain that only noise is being received. The calculation can be done adaptively, so that the system can react to changes in the noise.

A solution is now available for one of the unknowns in Equation 1. Another unknown, H₂(z), can be determined by using the instances where the VAD equals one and speech is being produced. When this is occurring, but the recent (perhaps less than 1 second) history of the microphones indicate low levels of noise, it can be assumed that n(s)=N(z)˜0. Then Equation 1 reduces to

M₁ₛ(z)=S(z)

M₂ₛ(z)=S(z)H₂(z)

which in turn leads to

$${M}_{2 s} ( z ) = {M}_{1 s} ( z ) {H}_{2} ( z )$$

$${H}_{2} ( z ) = \frac{{M}_{2 s} ( z )}{{M}_{1 s} ( z )}$$

which is the inverse of the H₁(z) calculation. However, it is noted that different inputs are being used—now only the signal is occurring whereas before only the noise was occurring. While calculating H₂(z), the values calculated for H₁(z) are held constant and vice versa. Thus, it is assumed that while one of H₁(z) and H₂(z) are being calculated, the one not being calculated does not change substantially.

After calculating H₁(z) and H₂(z), they are used to remove the noise from the signal. If Equation 1 is rewritten as

S(z)=M₁(z)−N(z)H₁(z)

N(z)=M₂(z)−S(z)H₂(z)

S(z)=M₁(z)−[M₂(z)−S(z)H₂(z)]H₁(z)′

S(z)[1−H₂(z)H₁(z)]=M₁(z)−M₂(z)H₁(z)

then N(z) may be substituted as shown to solve for S(z) as:

$$S ( z ) = \frac{{M}_{1} ( z ) - {M}_{2} ( z ) {H}_{1} ( z )}{1 - {H}_{2} ( z ) {H}_{1} ( z )} . \qquad Eq . 3$$

If the transfer functions H₁(z) and H₂(z) can be described with sufficient accuracy, then the noise can be completely removed and the original signal recovered. This remains true without respect to the amplitude or spectral characteristics of the noise. The only assumptions made are a perfect VAD, sufficiently accurate H₁(z) and H₂(z), and that when one of H₁(z) and H₂(z) are being calculated the other does not change substantially. In practice these assumptions have proven reasonable.

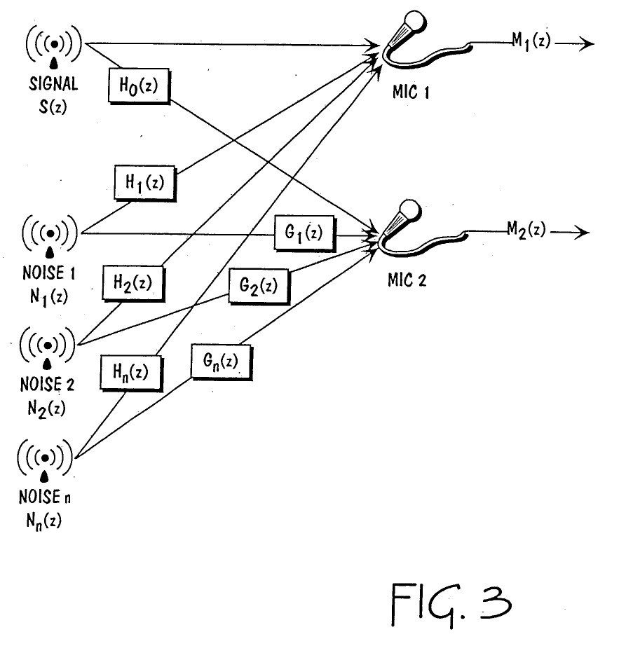

The noise removal algorithm described herein is easily generalized to include any number of noise sources. FIG. 3 is a block diagram of a front end of a noise removal algorithm of an embodiment, generalized to n distinct noise sources. These distinct noise sources may be reflections or echoes of one another, but are not so limited. There are several noise sources shown, each with a transfer function, or path, to each microphone. The previously named path H₂ has been relabeled as H₀, so that labeling noise source 2's path to MIC 1 is more convenient. The outputs of each microphone, when transformed to the z domain, are:

M₁(z)=S(z)+N₁(z)H₁(z)+N₂(z)H₂(z)+ . . . Nₙ(z)Hₙ(z)

M₂(z)=S(z)H₀(z)+N₁(z)G₁(z)+N₂(z)G₂(z)+ . . . Nₙ(z)Gₙ(z) Eq. 4

When there is no signal (VAD=0), then (suppressing the z's for clarity)

M₁ₙ=N₁H₁+N₂H₂+ . . . NₙHₙ

M₂ₙ=N₁G₁+N₂G₂+ . . . NₙGₙ Eq. 5

A new transfer function can now be defined, analogous to H₁(z) above:

$${\tilde{H}}_{1} = \frac{{M}_{1 n}}{{M}_{2 n}} = \frac{{N}_{1} {H}_{1} + {N}_{2} {H}_{2} + … {N}_{n} {H}_{n}}{{N}_{1} {G}_{1} + {N}_{2} {G}_{2} + … {N}_{n} {G}_{n}} \qquad Eq . 6$$

Thus \tilde{H}₁ depends only on the noise sources and their respective transfer functions and can be calculated any time there is no signal being transmitted. Once again, the n subscripts on the microphone inputs denote only that noise is being detected, while an s subscript denotes that only signal is being received by the microphones.

Examining Equation 4 while assuming that there is no noise produces

M₁ₛ=S

M₂ₛ=SH₀

Thus H₀ can be solved for as before, using any available transfer function calculating algorithm. Mathematically

$${H}_{0} = \frac{{M}_{2 s}}{{M}_{1 s}}$$

Rewriting Equation 4, using \tilde{H}₁ defined in Equation 6, provides,

$${\tilde{H}}_{1} = \frac{{M}_{1} - S}{{M}_{2} - {SH}_{0}} \qquad Eq . 7$$

Solving for S yields,

$$S = \frac{{M}_{1} - {M}_{2} {\tilde{H}}_{1}}{1 - {H}_{0} {\tilde{H}}_{1}} \qquad Eq . 8$$

which is the same as Equation 3, with H₀ taking the place of H₂, and \tilde{H}₁ taking the place of H₁. Thus the noise removal algorithm still is mathematically valid for any number of noise sources, including multiple echoes of noise sources. Again, if H₀ and \tilde{H}₁ can be estimated to a high enough accuracy, and the above assumption of only one path from the signal to the microphones holds, the noise may be removed completely.

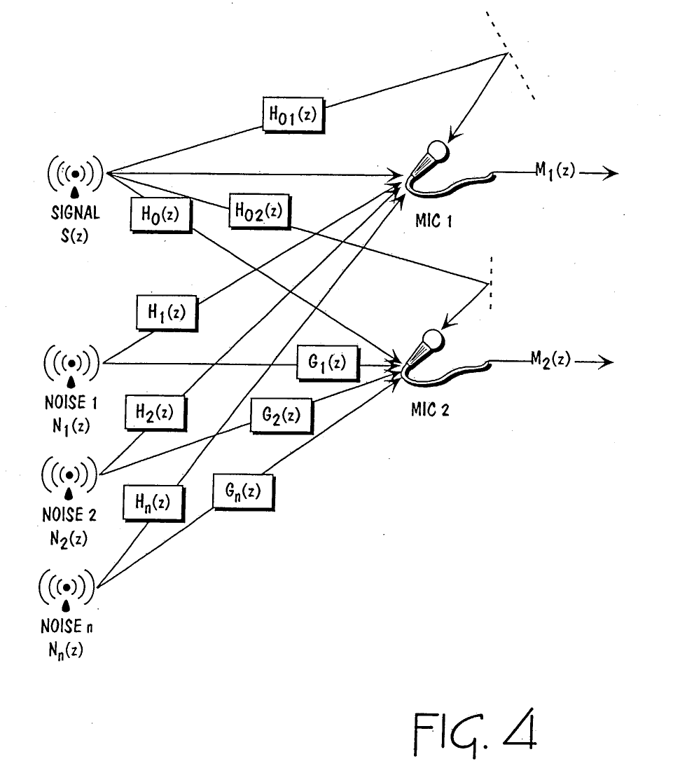

The most general case involves multiple noise sources and multiple signal sources. FIG. 4 is a block diagram of a front end of a noise removal algorithm of an embodiment in the most general case where there are n distinct noise sources and signal reflections. Here, reflections of the signal enter both microphones. This is the most general case, as reflections of the noise source into the microphones can be modeled accurately as simple additional noise sources. For clarity, the direct path from the signal to MIC 2 has changed from H₀(z) to H₀₀(z), and the reflected paths to MIC 1 and MIC 2 are denoted by H₀₁(z) and H₀₂(z), respectively.

The input into the microphones now becomes

M₁(z)=S(z)+S(z)H₀₁(z)+N₁(z)H₁(z)+N₂(z)H₂(z)+ . . . Nₙ(z)Hₙ(z)

M₂(z)=S(z)H₀₀(z)+S(z)H₀₂(z)+N₁(z)G₁(z)+N₂(z)G₂(z)+ . . . Nₙ(z)G₂(z) Eq. 9

When the VAD=0, the inputs become (suppressing the z's again)

M₁ₙ=N₁H₁+N₂H₂+ . . . NₙHₙ

M₂ₙ=N₁G₁+N₂G₂+ . . . NₙGₙ

which is the same as Equation 5. Thus, the calculation of \tilde{H}₁ in Equation 6 is unchanged, as expected. In examining the situation where there is no noise, Equation 9 reduces to

M₁ₛ=S+SH₀₁

M₂ₛ=SH₀₀+SH₀₂.

This leads to the definition of \tilde{H}₂:

$${\tilde{H}}_{2} = \frac{{M}_{2 s}}{{M}_{1 s}} = \frac{{H}_{00} + {H}_{02}}{1 + {H}_{01}} \qquad Eq . 10$$

Rewriting Equation 9 again using the definition for \tilde{H}₁ (as in Equation 7) provides

$${\tilde{H}}_{1} = \frac{{M}_{1} - S ( 1 + {H}_{01} )}{{M}_{2} - S ( {H}_{00} + {H}_{02} )} \qquad Eq . 11$$

Some algebraic manipulation yields

$$S ( 1 + {H}_{01} - {\tilde{H}}_{1} ( {H}_{00} + {H}_{02} ) ) = {M}_{1} - {M}_{2} {\tilde{H}}_{1} \\ S ( 1 + {H}_{01} ) [ 1 - {\tilde{H}}_{1} \frac{( {H}_{00} + {H}_{02} )}{( 1 + {H}_{01} )} ] = {M}_{1} - {M}_{2} {\tilde{H}}_{1} \\ S ( 1 + {H}_{01} ) [ 1 - {\tilde{H}}_{1} {\tilde{H}}_{2} ] = {M}_{1} - {M}_{2} {\tilde{H}}_{1} \\ and finally \\ S ( 1 + {H}_{01} ) = \frac{{M}_{1} - {M}_{2} {\tilde{H}}_{1}}{1 - {\tilde{H}}_{1} {\tilde{H}}_{2}} \qquad Eq . 12$$

Equation 12 is the same as equation 8, with the replacement of H₀ by \tilde{H}₂, and the addition of the (1+H₀₁) factor on the left side. This extra factor means that S cannot be solved for directly in this situation, but a solution can be generated for the signal plus the addition of all of its echoes. This is not such a bad situation, as there are many conventional methods for dealing with echo suppression, and even if the echoes are not suppressed, it is unlikely that they will affect the comprehensibility of the speech to any meaningful extent. The more complex calculation of \tilde{H}₂ is needed to account for the signal echoes in MIC 2, which act as noise sources.

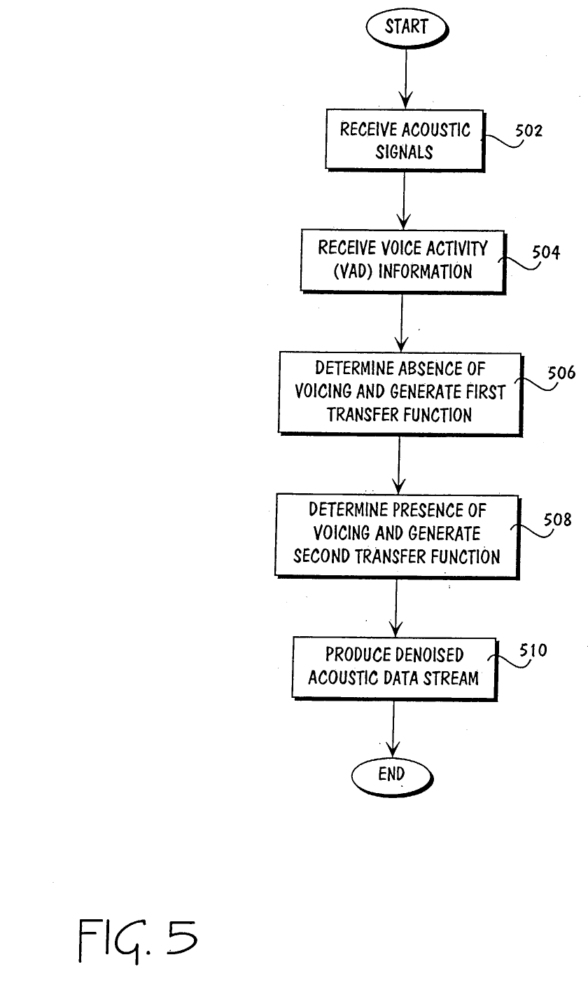

FIG. 5 is a flow diagram of a denoising method of an embodiment. In operation, the acoustic signals are received 502. Further, physiological information associated with human voicing activity is received 504. A first transfer function representative of the acoustic signal is calculated upon determining that voicing information is absent from the acoustic signal for at least one specified period of time 506. A second transfer function representative of the acoustic signal is calculated upon determining that voicing information is present in the acoustic signal for at least one specified period of time 508. Noise is removed from the acoustic signal using at least one combination of the first transfer function and the second transfer function, producing denoised acoustic data streams 510.

An algorithm for noise removal, or denoising algorithm, is described herein, from the simplest case of a single noise source with a direct path to multiple noise sources with reflections and echoes. The algorithm has been shown herein to be viable under any environmental conditions. The type and amount of noise are inconsequential if a good estimate has been made of \tilde{H}₁ and \tilde{H}₂, and if one does not change substantially while the other is calculated. If the user environment is such that echoes are present, they can be compensated for if coming from a noise source. If signal echoes are also present, they will affect the cleaned signal, but the effect should be negligible in most environments.

In operation, the algorithm of an embodiment has shown excellent results in dealing with a variety of noise types, amplitudes, and orientations. However, there are always approximations and adjustments that have to be made when moving from mathematical concepts to engineering applications. One assumption is made in Equation 3, where H₂(z) is assumed small and therefore H₂(z)H₁(z)≈0, so that Equation 3 reduces to

S(z)≈M₁(z)−M₂(z)H₁(z).

This means that only H₁(z) has to be calculated, speeding up the process and reducing the number of computations required considerably. With the proper selection of microphones, this approximation is easily realized.

Another approximation involves the filter used in an embodiment. The actual H₁(z) will undoubtedly have both poles and zeros, but for stability and simplicity an all-zero Finite Impulse Response (FIR) filter is used. With enough taps (around 60) the approximation to the actual H₁(z) is very good.

Regarding subband selection, the wider the range of frequencies over which a transfer function must be calculated, the more difficult it is to calculate it accurately. Therefore the acoustic data was divided into 16 subbands, with the lowest frequency at 50 Hz and the highest at 3700. The denoising algorithm was then applied to each subband in turn, and the 16 denoised data streams were recombined to yield the denoised acoustic data. This works very well, but any combinations of subbands (i.e. 4, 6, 8, 32, equally spaced, perceptually spaced, etc.) can be used and has been found to work as well.

The amplitude of the noise was constrained in an embodiment so that the microphones used did not saturate (that is, operate outside a linear response region). It is important that the microphones operate linearly to ensure the best performance. Even with this restriction, very low signal-to-noise ratio (SNR) signals can be denoised (down to −10 dB or less).

The calculation of H₁(z) is accomplished every 10 milliseconds using the Least-Mean Squares (LMS) method, a common adaptive transfer function. An explanation may be found in “Adaptive Signal Processing” (1985), by Widrow and Steams, published by Prentice-Hall, ISBN 0-13-004029-0.

The VAD for an embodiment is derived from a radio frequency sensor and the two microphones, yielding very high accuracy (>99%) for both voiced and unvoiced speech. The VAD of an embodiment uses a radio frequency (RF) interferometer to detect tissue motion associated with human speech production, but is not so limited. It is therefore completely acoustic-noise free, and is able to function in any acoustic noise environment. A simple energy measurement of the RF signal can be used to determine if voiced speech is occurring. Unvoiced speech can be determined using conventional acoustic-based methods, by proximity to voiced sections determined using the RF sensor or similar voicing sensors, or through a combination of the above. Since there is much less energy in unvoiced speech, its activation accuracy is not as critical as voiced speech.

With voiced and unvoiced speech detected reliably, the algorithm of an embodiment can be implemented. Once again, it is useful to repeat that the noise removal algorithm does not depend on how the VAD is obtained, only that it is accurate, especially for voiced speech. If speech is not detected and training occurs on the speech, the subsequent denoised acoustic data can be distorted.

Data was collected in four channels, one for MIC 1, one for MIC 2, and two for the radio frequency sensor that detected the tissue motions associated with voiced speech. The data were sampled simultaneously at 40 kHz, then digitally filtered and decimated down to 8 kHz. The high sampling rate was used to reduce any aliasing that might result from the analog to digital process. A four-channel National Instruments A/D board was used along with Labview to capture and store the data. The data was then read into a C program and denoised 10 milliseconds at a time.

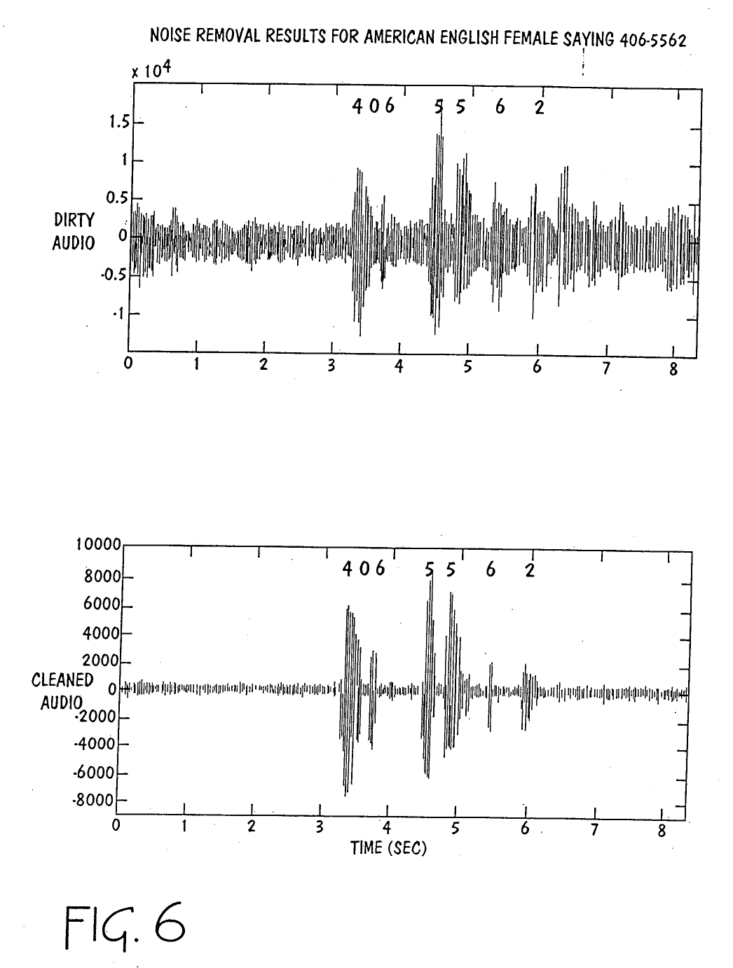

FIG. 6 shows results of a noise suppression algorithm of an embodiment for an American English speaking female in the presence of airport terminal noise that includes many other human speakers and public announcements. The speaker is uttering the numbers 406-5562 in the midst of moderate airport terminal noise. The dirty acoustic data was denoised 10 milliseconds at a time, and before denoising the 10 milliseconds of data were prefiltered from 50 to 3700 Hz. A reduction in the noise of approximately 17 dB is evident. No post filtering was done on this sample; thus, all of the noise reduction realized is due to the algorithm of an embodiment. It is clear that the algorithm adjusts to the noise instantly, and is capable of removing the very difficult noise of other human speakers. Many different types of noise have all been tested with similar results, including street noise, helicopters, music, and sine waves, to name a few. Also, the orientation of the noise can be varied substantially without significantly changing the noise suppression performance. Finally, the distortion of the cleaned speech is very low, ensuring good performance for speech recognition engines and human receivers alike.

The noise removal algorithm of an embodiment has been shown to be viable under any environmental conditions. The type and amount of noise are inconsequential if a good estimate has been made of \tilde{H}₁ and \tilde{H}₂. If the user environment is such that echoes are present, they can be compensated for if coming from a noise source. If signal echoes are also present, they will affect the cleaned signal, but the effect should be negligible in most environments.

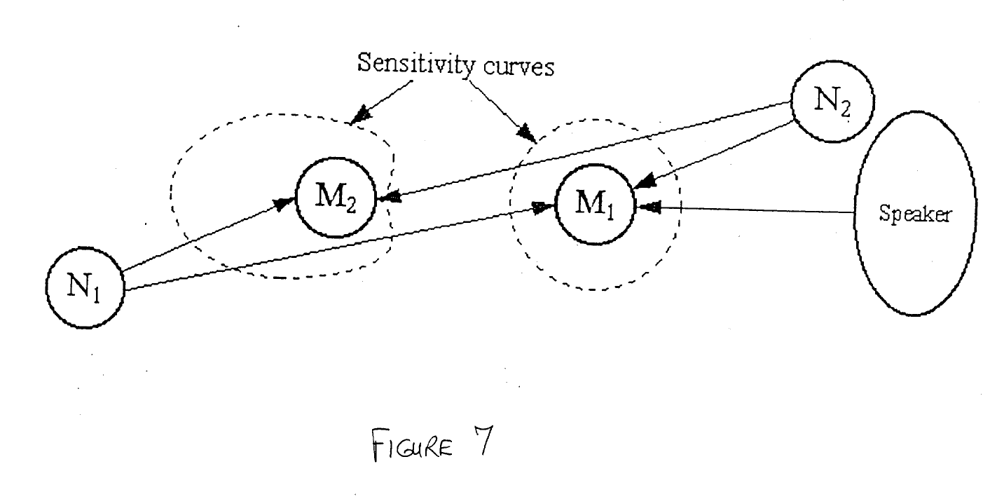

FIG. 7 is a block diagram of a physical configuration for denoising using a unidirectional microphone M2 for the noise and an omnidirectional microphone M1 for the speech, under the embodiments of FIGS. 2, 3, and 4. As described above, the path from the speech to the noise microphone (MIC 2) is approximated as zero, and that approximation is realized through the careful placement of omnidirectional and unidirectional microphones. This works quite well (20-40 dB of noise suppression) when the noise is oriented opposite the signal location (noise source N₁). However, when the noise source is oriented on the same side as the speaker (noise source N₂), the performance can drop to only 10-20 dB of noise suppression. This drop in suppression ability can be attributed to the steps taken to ensure that H₂ is close to zero. These steps included the use of a unidirectional microphone for the noise microphone (MIC 2) so that very little signal is present in the noise data. As the unidirectional microphone cancels out acoustic information coming from a particular direction, it also cancels out noise that is coming from the same direction as speech. This may limit the ability of the adaptive algorithm to characterize and then remove noise in a location such as N₂. The same effect is noted when a unidirectional microphone is used for the speech microphone, M1.

However, if the unidirectional microphone M₂ is replaced with an omnidirectional microphone, then a significant amount of signal is captured by M₂. This runs counter to the aforementioned assumption that H₂ is zero, and as a result during voicing a significant amount of signal is removed, resulting in denoising and “de-signaling”. This is not acceptable if signal distortion is to be kept to a minimum. In order to reduce the distortion, therefore, a value is calculated for H₂. However, the value for H₂ can not be calculated in the presence of noise, or the noise will be mislabeled as speech and not removed.

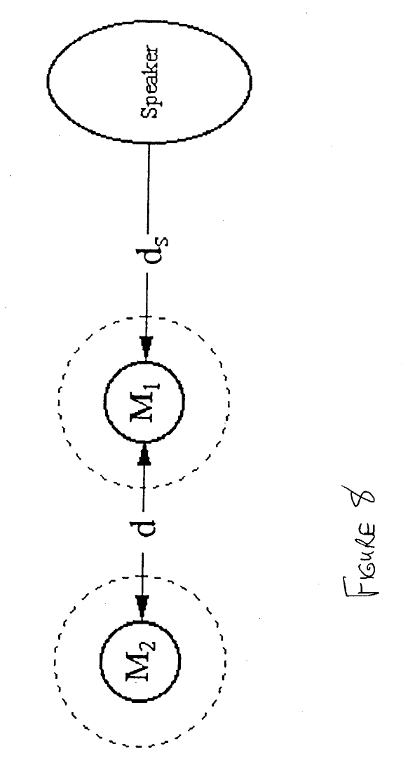

Experience with acoustic-only microphone arrays suggests that a small, two-microphone array might be a solution to the problem. FIG. 8 is a denoising microphone configuration including two omnidirectional microphones, under an embodiment. The same effect can be achieved through the use of two unidirectional microphones, oriented in the same direction (toward the signal source). Yet another embodiment uses one unidirectional microphone and one omnidirectional microphone. The idea is to capture similar information from acoustic sources in the direction of the signal source. The relative locations of the signal source and the two microphones are fixed and known. By placing the microphones a distance d apart that corresponds with n discrete time samples and placing the speaker on the axis of the array, H₂ can be fixed to be of the form Cz⁻ⁿ, where C is the difference in amplitude of the signal data at M₁ and M₂. For the discussion that follows, the assumption is made that n=1, although any integer other than zero may be used. For causality, the use of positive integers is recommended. As the amplitude of a spherical pressure source varies as 1 /r, this allows not only specification of the direction of the source but its distance. The C required can be estimated by

$$C = \frac{\vert S \vert at {M}_{2}}{\vert S \vert at {M}_{1}} ∝ \frac{{d}_{s}}{d + {d}_{s}} .$$

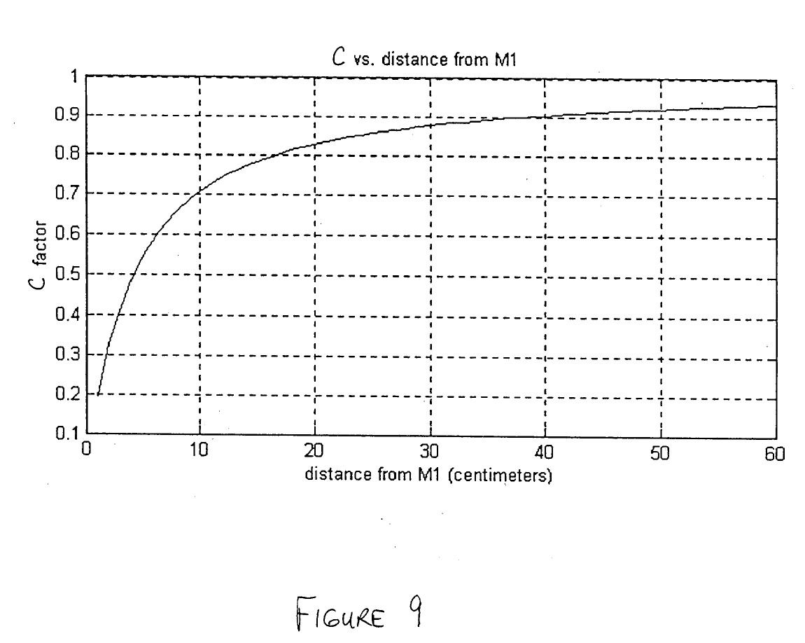

FIG. 9 is a plot of the C required versus distance, under the embodiment of FIG. 8. It can be seen that the asymptote is at C=1.0, and C reaches 0.9 at approximately 38 centimeters, slightly more than a foot, and 0.94 at approximately 60 cm. At the distances normally encountered in a handset and earpiece (4 to 12 cm), C would be between approximately 0.5 to 0.75. This is a difference of approximately 19 to 44% with the noise source located at approximately 60 cm, and it is clear that most noise sources would be located farther away than that. Therefore, the system using this configuration would be able to discriminate between noise and signal quite effectively, even when they have a similar orientation.

To determine the effects on denoising of poor estimates of C, assume that C=nC₀, where C is an estimate and C₀ is the actual value of C. Using the signal definition from above,

$$S ( z ) = \frac{{M}_{1} ( z ) - {M}_{2} ( z ) {H}_{1} ( z )}{1 - {H}_{2} ( z ) {H}_{1} ( z )} ,$$

it has been assumed that H₂(z) was very small, so that the signal could be approximated by

S(z)≈M₁(z)−M₂(z)H₁(z).

This is true if there is no speech, because by definition H₂=0. However, if speech is occurring, H₂ is nonzero, and if set to be Cz⁻¹,

$$S ( z ) = \frac{{M}_{1} ( z ) - {M}_{2} ( z ) {H}_{1} ( z )}{1 - C {z}^{- 1} {H}_{1} ( z )} ,$$

which can be rewritten as

$$S ( z ) = \frac{{M}_{1} ( z ) - {M}_{2} ( z ) {H}_{1} ( z )}{1 - n {C}_{0} {z}^{- 1} {H}_{1} ( z )} = \frac{{M}_{1} ( z ) - {M}_{2} ( z ) {H}_{1} ( z )}{1 - {C}_{0} {z}^{- 1} {H}_{1} ( z ) + ( 1 - n ) {C}_{0} {z}^{- 1} {H}_{1} ( z )} .$$

The last factor in the denominator determines the error due to the poor estimation of C. This factor is labeled E:

E=(1−n)C₀z⁻¹H₁(z).

Because z⁻¹H₁(z) is a filter, its magnitude will always be positive. Therefore the change in calculated signal magnitude due to E will depend completely on (1−n).

There are two possibilities for errors: underestimation of C (n<1), and overestimation of C (n>1). In the first case, C is estimated to be smaller that it actually is, or the signal is closer than estimated. In this case (1−n) and therefore E is positive. The denominator is therefore too large, and the magnitude of the cleaned signal is too small. This would indicate de-signaling. In the second case, the signal is farther away than estimated, and E is negative, making S larger than it should be. In this case the denoising is insufficient. Because very low signal distortion is desired, the estimations should err toward overestimation of C.

This result also shows that noise located in the same solid angle (direction from M₁) as the signal will be substantially removed depending on the change in C between the signal location and the noise location. Thus, when using a handset with M₁ approximately 4 cm from the mouth, the required C is approximately 0.5, and for noise at approximately 1 meter the C is approximately 0.96. Thus, for the noise, the estimate of C=0.5 means that for the noise C is underestimated, and the noise will be removed. The amount of removal will depend directly on (1−n). Therefore, this algorithm uses the direction and the range to the signal to separate the signal from the noise.

One issue that arises involves stability of this technique. Specifically, the deconvolution of (1 −H₁H₂) raises the question of stability, as the need arises to calculate the inverse of 1 −H₁H₂ at the beginning of each voiced segment. This helps reduce the computing time, or number of instructions per cycle, needed to implement the algorithm, as there is no requirement to calculate the inverse for every voiced window, just the first one, as H₂ is considered to be constant. This approximation will make false positives more computationally expensive, however, by requiring a calculation of the inverse of 1−H₁H₂ every time a false positive is encountered.

Fortunately, the choice of H₂ eliminates the need for a deconvolution. From the discussion above, the signal can be written as

$$S ( z ) = \frac{{M}_{1} ( z ) - {M}_{2} ( z ) {H}_{1} ( z )}{1 - {H}_{2} ( z ) {H}_{1} ( z )} ,$$

which can be rewritten as

S(z)=M₁(z)−M₂(z)H₁(z)+S(z)H₂(z)H₁(z),

or

S(z)=M₁(z)−H₁(z)[M₂(z)+S(z)H₂(z)].

However, since H₂(z) is of the form Cz⁻¹, the sequence in the time domain would look like

s[n]=m₁[n]−h₁*[m₂[n]−C·s[n−1]],

meaning that the present signal sample requires the present MIC 1 signal, the present MIC 2 signal, and the previous signal sample. This means that no deconvolution is needed, just a simple subtraction and then a convolution as before. The increase in computations required is minimal. Therefore this improvement is easy to implement.

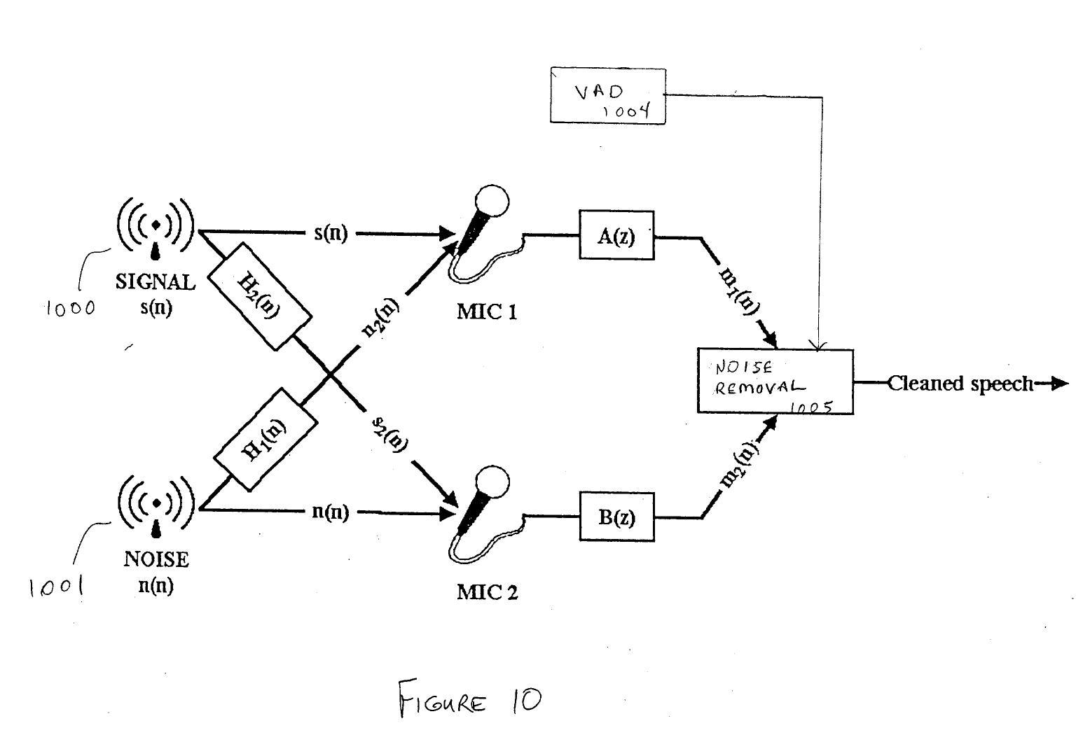

The effects of the difference in microphone response on this embodiment can be shown by examining the configurations described with reference to FIGS. 2, 3, and 4, only this time transfer functions A(z) and B(z) are included, which represent the frequency response of MIC 1 and MIC 2 along with their filtering and amplification responses. FIG. 10 is a block diagram of a front end of a noise removal algorithm under an embodiment in which the two microphones MIC 1 and MIC 2 have different response characteristics.

FIG. 10 includes a graphic description of the process of an embodiment, with a single signal source 1000 and a single noise source 1001. This algorithm uses two microphones: a “signal” microphone 1 (“MIC1”) and a “noise” microphone 2 (“MIC 2”), but is not so limited. MIC 1 is assumed to capture mostly signal with some noise, while MIC 2 captures mostly noise with some signal. The data from the signal source 1000 to MIC 1 is denoted by s(n), where s(n) is a discrete sample of the analog signal from the source 1000. The data from the signal source 1000 to MIC 2 is denoted by s₂(n). The data from the noise source 1001 to MIC 2 is denoted by n(n). The data from the noise source 1001 to MIC 1 is denoted by n₂(n).

A transfer functions A(z) represents the frequency response of MIC 1 along with its filtering and amplification responses. A transfer function B(z) represents the frequency response of MIC 2 along with its filtering and amplification responses. The output of the transfer function A(z) is denoted by m₁(n), and the output of the transfer function B(z) is denoted by m₂(n). The signal m₁(n) and m₂(n) are received by a noise removal element 1005, which operates on the signals and outputs “cleaned speech”.

Hereafter, the term “frequency response of MIC X” will include the combined effects of the microphone and any amplification or filtering processes that occur during the data recording process for that microphone. When solving for the signal and noise (suppressing “z” for clarity),

$$S = \frac{{M}_{1}}{A} - {H}_{1} N \\$$

$$N = \frac{{M}_{2}}{B} - {H}_{2} S$$

wherein substituting the latter into the former produces

$$S = \frac{{M}_{1}}{A} - \frac{{H}_{1} {M}_{2}}{B} + {H}_{1} {H}_{2} S \\$$

$$S = \frac{\frac{{M}_{1}}{A} - \frac{{H}_{1} {M}_{2}}{B}}{1 - {H}_{1} {H}_{2}}$$

which seems to indicate that the differences in frequency response (between MIC 1 and MIC 2) have an impact. However, what is being measured has to be noted. Formerly (before taking the frequency response of the microphones into account), H₁ was measured using

$${H}_{1} = \frac{{M}_{1 n}}{{M}_{2 n}} ,$$

where the n subscripts indicate that this calculation only occurs during windows that contain only noise. However, when examining the equations, it is noted that when there is no signal the following is measured at the microphones:

M₁=H₁NA

M₂=NB

therefore H₁ should be calculated as

$${H}_{1} = \frac{B {M}_{1 n}}{A {M}_{2 n}} .$$

However, B(z) and A(z) are not taken into account when calculating H₁(z). Therefore what is actually measured is just the ratio of the signals in each microphone:

$${\tilde{H}}_{1} = \frac{{M}_{1 n}}{{M}_{2 n}} = {H}_{1} \frac{A}{B} ,$$

where \tilde{H}₁ represents the measured response and H₁ the actual response. The calculation for H₂ is analogous, and results in

$${\tilde{H}}_{2} = \frac{{M}_{2 s}}{{M}_{1 s}} = {H}_{2} \frac{B}{A} .$$

Substituting \tilde{H}₁ and \tilde{H}₂ back into the equation for S above produces

$$S = \frac{\frac{{M}_{1}}{A} - \frac{B {\tilde{ H}}_{1} {M}_{2}}{AB}}{1 - {\tilde{H}}_{1} \frac{B}{A} {\tilde{H}}_{2} \frac{A}{B}} , or SA = \frac{{M}_{1} - {\tilde{H}}_{1} {M}_{2}}{1 - {\tilde{H}}_{1} {\tilde{H}}_{2}} ,$$

which is the same as before, when the frequency response of the microphones was not included. Here S(z)A(z) takes the place of S(z), and the values (\tilde{H}₁(z) and \tilde{H}₂ (z)) take the place of the actual H₁(z) and H₂(z). Thus, this algorithm is, in theory, independent of the microphone and associated filter and amplifier response.

However, in practice, it is assumed that H₂=Cz⁻¹ (where C is a constant), but it is actually

$${\tilde{H}}_{2} = \frac{B}{A} {Cz}^{- 1}$$

$$\text{so the result is}$$

$$SA = \frac{{M}_{1} - {\tilde{H}}_{1} {M}_{2}}{1 - \frac{B}{A} {\tilde{H}}_{1} {Cz}^{- 1}} ,$$

which is dependent on B(z) and A(z), which are not known. This can cause problems if the frequency response of the microphones is substantially different, which is a common occurrence, especially for the inexpensive microphones frequently used. This means that the data from MIC 2 should be compensated so that it has the proper relationship to the data coming from MIC1. This can be done by recording a broadband signal in both MIC 1 and MIC 2 from a source that is located at the distance and orientation expected for the actual signal (the actual signal source could also be used). A discrete Fourier transform (DFT) for each microphone signal is then calculated, and the magnitude of the transform at each frequency bin is calculated. The magnitude of the DFT for MIC 2 in each frequency bin is then set to be equal to C multiplied by the magnitude of the DFT for MIC 1. If M₁[n] represents the n^{th} frequency bin magnitude of the DFT for MIC 1, then the factor that is multiplied by M₂[n] would be

$$F [ n ] = C \frac{{M}_{1} [ n ]}{{M}_{2} [ n ]}$$

The inverse transform is then applied to the new MIC 2 DFT amplitude, using the previous MIC 2 DFT phase. In this manner, MIC 2 is resynthesized so that the relationship

M₂(z)=M₁(z)·Cz⁻¹

is correct for the times when only speech is occurring. This transformation could also be performed in the time domain, using a filter that would emulate the properties of F as closely as possible (for example, the Matlab function FFT2.M could be used with the calculated values of F[n] to construct a suitable FIR filter).

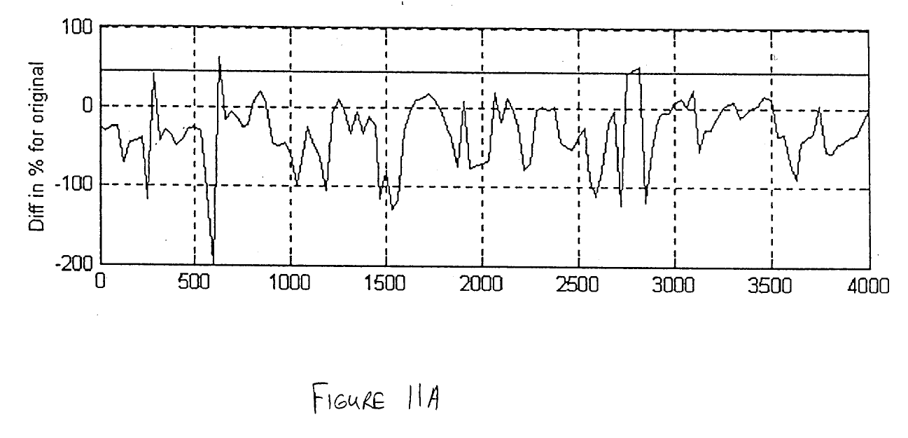

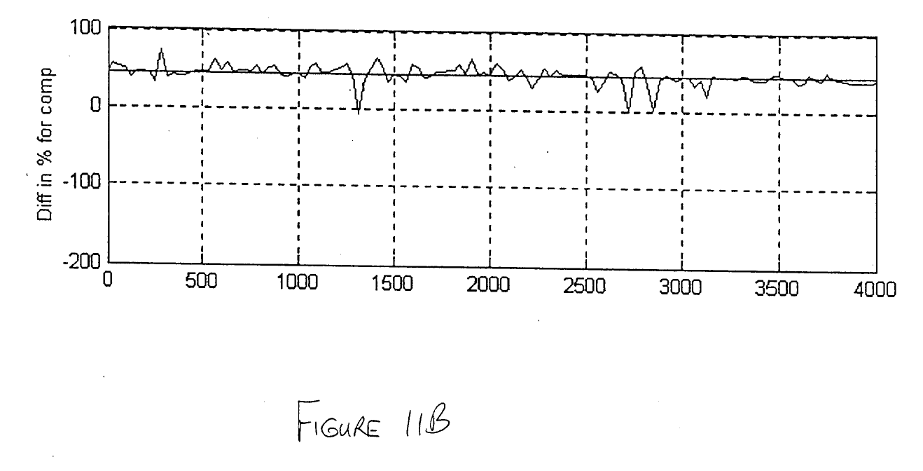

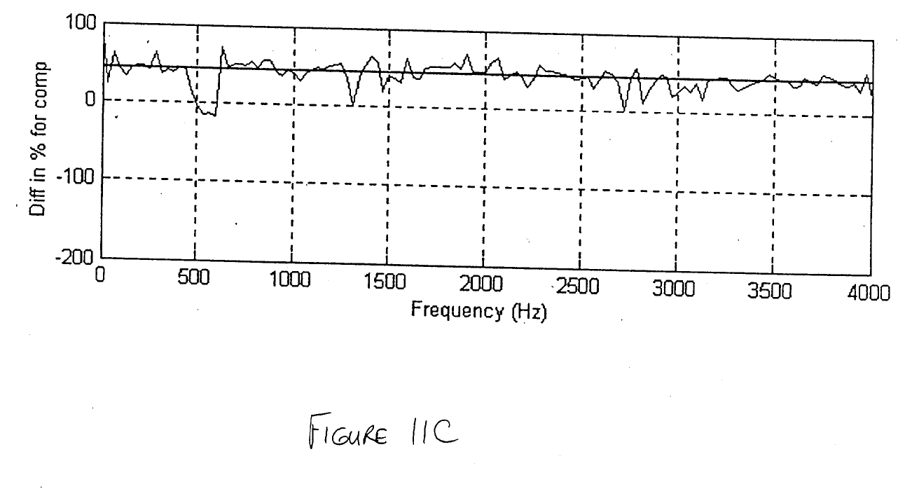

FIG. 11A is a plot of the difference in frequency response (percent) between the microphones (at a distance of 4 centimeters) before compensation. FIG. 11B is a plot of the difference in frequency response (percent) between the microphones (at a distance of 4 centimeters) after DFT compensation. FIG. 11C is a plot of the difference in frequency response (percent) between the microphones (at a distance of 4 centimeters) after time-domain filter compensation. These plots show the effectiveness of the compensation methods described above. Thus, using two very inexpensive omnidirectional or unidirectional microphones, both compensation methods restore the correct relationship between the microphones.

The transformation should be relatively constant as long as the relative amplifications and filtering processes are unchanged. Thus, it is possible that the compensation process would only need to be performed once at the manufacturing stage. However, if need be, the algorithm could be set to operate assuming H₂=0 until the system was used in a place with very little noise and strong signal. Then the compensation coefficients F[n] could be calculated and used from that time on. Since denoising is not required when there is very little noise, this calculation would not impose undue strain on the denoising algorithm. The denoising coefficients could also be updated any time the noise environment is favorable for maximum accuracy.

Each of the blocks and steps depicted in the figures presented herein can each include a sequence of operations that need not be described herein. Those skilled in the relevant art can create routines, algorithms, source code, microcode, program logic arrays or otherwise implement the invention based on the figures and the detailed description provided herein. The routines described herein can include any of the following, or one or more combinations of the following: a routine stored in non-volatile memory (not shown) that forms part of an associated processor or processors; a routine implemented using conventional programmed logic arrays or circuit elements; a routine stored in removable media such as disks; a routine downloaded from a server and stored locally at a client; and a routine hardwired or preprogrammed in chips such as electrically erasable programmable read only memory (“EEPROM”) semiconductor chips, application specific integrated circuits (ASICs), or by digital signal processing (DSP) integrated circuits.

Unless the context clearly requires otherwise, throughout the description and the claims, the words “comprise,” “comprising,” and the like are to be construed in an inclusive sense as opposed to an exclusive or exhaustive sense; that is to say, in a sense of “including, but not limited to.” Words using the singular or plural number also include the plural or singular number respectively. Additionally, the words “herein,” “hereunder,” and words of similar import, when used in this application, shall refer to this application as a whole and not to any particular portions of this application.

The above description of illustrated embodiments of the invention is not intended to be exhaustive or to limit the invention to the precise form disclosed. While specific embodiments of, and examples for, the invention are described herein for illustrative purposes, various equivalent modifications are possible within the scope of the invention, as those skilled in the relevant art will recognize. The teachings of the invention provided herein can be applied to other machine vision systems, not only for the data collection symbology reader described above. Further, the elements and acts of the various embodiments described above can be combined to provide further embodiments.

Any references or U.S. patent applications referenced herein are incorporated herein by reference. Aspects of the invention can be modified, if necessary, to employ the systems, functions and concepts of these various references to provide yet further embodiments of the invention.

What is claimed is:

1. A method for removing noise from electronic signals, comprising:
receiving a plurality of acoustic signals in a first receiving device;
receiving a plurality of acoustic signals in a second receiving device, wherein the plurality of acoustic signals include at least one noise signal generated by at least one noise source and at least one voice signal generated by at least one signal source, wherein the at least one signal source comprises a human speaker, and wherein relative locations of the signal source, the first receiving device, and the second receiving device are fixed and known;
receiving physiological information associated with human voicing activity of the human speaker, including whether voice activity is present;
generating at least one first transfer function representative of the plurality of acoustic noise signals upon determining that voicing activity is absent from the plurality of acoustic signals for at least one specified period;
generating at least one second transfer function representative of the plurality of acoustic signals upon determining that voicing information is present in the plurality of acoustic signals for the at least one specified period of time; and
removing noise from the plurality of acoustic signals using at least one combination of the at least one first transfer function and the at least one second transfer function to produce at least one denoised data stream.

2. The method of claim 1, wherein the first receiving device and the second receiving device each comprise a microphone selected from a group comprising unidirectional microphones and unidirectional microphones.

3. The method of claim 1, wherein the plurality of acoustic signals are received in discrete time samples, and wherein the first receiving device and the second receiving device are located a distance “d” apart, wherein d corresponds to n discrete time samples

4. The method of claim 1, wherein the at least one second transfer function is fixed as a function of a difference in amplitude of signal data at the first receiving device and the amplitude of signal data at the second receiving device.

5. The method of claim 1, wherein removing noise from the plurality of acoustic signals includes using a direction and a range to the at least one signal source from the at least one first receiving device.

6. The method of claim 1, wherein respective frequency responses of the at least one first receiving device and the second at least one receiving device are different, and wherein the signal data from the at least one second receiving device is compensated to have a proper relationship to signal data from the at least one first receiving device.

7. The method of claim 6, wherein compensating the signal data from the at least one second receiving device comprises recording a broadband signal in the at least one first receiving device and the at least one second receiving device from a source located at a distance and an orientation expected for a signal from the at least one signal source.

8. The method of claim 6, wherein compensating the signal data from the at least one second receiving device comprises frequency domain compensation.

9. The method of claim 8, wherein frequency compensation comprises:
calculating a frequency transform for signal data from each of the at least one first receiving device and the at least one second receiving device signal is calculated;
calculating a magnitude of the frequency transform at each frequency bin; and
setting a magnitude of the frequency transform for the signal data from the at least one second receiving device in each frequency to a value related to a magnitude of the frequency transform for the signal data from the at least one first receiving device.

10. The method of claim 6, wherein compensating the signal data from the at least one second receiving device comprises time domain compensation.

11. The method of claim 6, further comprising:
initially setting the at least one second transfer function to zero; and
calculating compensation coefficients at times when there the at least one noise signal is small relative to the at least one voice signal.

12. The method of claim 1, wherein the plurality of acoustic signals include at least one reflection of the at least one noise signal and at least one reflection of the at least one voice signal.

13. The method of claim 1, wherein receiving physiological information comprises receiving physiological data associated with human voicing using at least one detector selected from a group consisting of acoustic microphones, radio frequency devices, electroglottographs, ultrasound devices, acoustic throat microphones, and airflow detectors.

14. The method of claim 1 wherein generating the at least one first transfer function and the at least one second transfer function comprises use of at least one technique selected from a group comprising adaptive techniques and recursive techniques.

15. A system for removing noise from acoustic signals, comprising:
at least one receiver comprising,
at least one signal receiver configured to receive at least one acoustic signal from a signal source; and
at least one noise receiver configured to receive at least one noise signal from a noise source, wherein relative locations of the signal source, the at lease one signal receiver, and the at least one noise receiver are fixed and known;
at least one sensor that receives physiological information associated with human voicing activity; and
at least one processor coupled among the at least one receiver and the at least one sensor that generates a plurality of transfer functions, wherein at least one first transfer function representative of the at least one acoustic signal is generated in response to a determination that voicing information is absent from the at least one acoustic signal for at least one specified period of time, wherein at least one second transfer function representative of the at least one acoustic signal is generated in response to a determination that voicing information is present in the at least one acoustic signal for at least one specified period of time, wherein noise is removed from the at least one acoustic signal using at least one combination of the at least one first transfer function and the at least one second transfer function.

16. The system of claim 15, wherein the at least one sensor includes at least one radio frequency (“RF”) interferometer that detects tissue motion associated with human speech.

17. The system of claim 15, wherein the at least one sensor includes at least one sensor selected from a group consisting of acoustic microphones, radio frequency devices, electroglottographs, ultrasound devices, acoustic throat microphones, and airflow detectors.

18. The system of claim 15, wherein the at least one processor is configured to:
divide acoustic data of the at least one acoustic signal into a plurality of subbands;
remove noise from each of the plurality of subbands using the at least one combination of the at least one first transfer function and the at least one second transfer function, wherein a plurality of denoised acoustic data streams are generated; and
combine the plurality of denoised acoustic data streams to generate the at least one denoised acoustic data stream.

19. The system of claim 15, wherein the at least one signal receiver and the at least one noise receiver are each microphones selected from a group comprising unidirectional microphones and omnidirectional microphones.

20. A signal processing system coupled among at least one user and at least one electronic device, the signal processing system comprising:
at least one first receiving device configured to receive at least one acoustic signal from a signal source;
at least one second receiving device configured to receive at least one noise signal from a noise source, wherein relative locations of the signal source, the at least one first receiving device, and the at least one second receiving device are fixed and known; and
at least one denoising subsystem for removing noise from acoustic signals, the denoising subsystem comprising:
at least one processor coupled among the at least one first receiver and the at least one second receiver; and
at least one sensor coupled to the at least one processor, wherein the at least one sensor is configures to receive physiological information associated with human voicing activity, wherein the at least one processor generates a plurality of transfer functions, wherein at least one first transfer function representative of the at least one acoustic signal is generated in response to a determination that voicing information is absent from the at least one acoustic signal for at least one specified period of time, wherein at least one second transfer function representative of the at least one acoustic signal is generated in response to a determination that voicing information is present in the at least one acoustic signal for at least one specified period of time, wherein noise is removed from the at least one acoustic signal using at least one combination of the at least one first transfer function and the at least one second transfer function to produce at least one denoised data stream.

21. The signal processing system of claim 20, wherein the first receiving device and the second receiving device are each microphones selected from a group comprising unidirectional microphones and omnidirectional microphones.

22. The signal processing system of claim 20, wherein the at least one acoustic signal is received in discrete time samples, and wherein the first receiving device and the second receiving device are located a distance “d” apart, wherein d corresponds to n discrete time samples

23. The signal processing system of claim 20, wherein the at least one second transfer function is fixed as a function of a difference in amplitude of signal data at the first receiving device and the amplitude of signal data at the second receiving device.

24. The signal processing system of claim 20, wherein removing noise from the at least one acoustic signal includes using a direction and a range to the at least one signal source from the at least one first receiving device.

25. The signal processing system of claim 20, wherein respective frequency responses of the at least one first receiving device and the second at least one receiving device are different, and wherein the signal data from the at least one second receiving device is compensated to have a proper relationship to signal data from the at least one first receiving device.

26. The signal processing system of claim 25, wherein compensating the signal data from the at least one second receiving device comprises recording a broadband signal in the at least one first receiving device and the at least one second receiving device from a source located at a distance and an orientation expected for a signal from the at least one signal source.

27. The signal processing system of claim 25, wherein compensating the signal data from the at least one second receiving device comprises frequency domain compensation.

28. The signal processing system of claim 27, wherein frequency compensation comprises:
calculating a frequency transform for signal data from each of the at least one first receiving device and the at least one second receiving device signal is calculated;
calculating a magnitude of the frequency transform at each frequency bin; and
setting a magnitude of the frequency transform for the signal data from the at least one second receiving device in each frequency to a value related to a magnitude of the frequency transform for the signal data from the at least one first receiving device.

29. The signal processing system of claim 25, wherein compensating the signal data from the at least one second receiving device comprises time domain compensation.

30. The signal processing system of claim 25, further compensating further comprises:
initially setting the at least one second transfer function to zero; and
calculating compensation coefficients at times when there the at least one noise signal is small relative to the at least one acoustic signal.

31. The signal processing system of claim 20, wherein the at least one acoustic signal includes at least one reflection of the at least one noise signal and at least one reflection of the at least one acoustic signal.

32. The signal processing system of claim 20, wherein receiving physiological information comprises receiving physiological data associated with human voicing using at least one detector selected from a group consisting of acoustic microphones, radio frequency devices, electroglottographs, ultrasound devices, acoustic throat microphones, and airflow detectors.

33. The signal processing system of claim 20 wherein generating the at least one first transfer function and the at least one second transfer function comprises use of at least one technique selected from a group comprising adaptive techniques and recursive techniques.
